(19) United States
(12) Patent Application Publication
(10) Pub. No.: US 2017/0011178 A1
(43) Pub. Date: Jan. 12, 2017

(54) SMART PHONE BASED MULTI-PATIENT WORKLIST (SPWL)

(71) Applicant: KONINKLIJKE PHILIPS N.V., Eindhoven (NL)

(72) Inventors: Maria F. HENDRICKSON, CHELMSFORD, MA (US); Andrew J. BAMBURY, ANDOVER, MA (US); James W. BRUETSCH, CHELMSFORD, MA (US); Rahul J. SHAH, WESTBOROUGH, MA (US); John SILVA, READING, MA (US); Clifford STRAW, ANDOVER, MA (US)

(21) Appl. No.: 15/122,204

(22) PCT Filed: Mar. 30, 2015
(86) PCT No.: PCT/IB2015/052325

§ 371 Date: Aug. 29, 2016

Related U.S. Application Data
(60) Provisional application No. 61/971,556, filed on Mar. 28, 2014.

(51) Int. Cl. G06F 19/00; G06Q 10/06

(52) U.S. Cl. CPC G06F 19/327; G06F 19/324; G06Q 10/063118

(57) ABSTRACT

When providing a smart phone based multi-patient worklist (SPWL), a smartphone application, or “app,” is provided for nurses or other healthcare providers to plan and coordinate the care of their assigned patients. The SPWL includes time-based and patient-based screens that allow a nurse to determine what needs to be done for a particular patient as well as what needs to be done at a particular time or time range for a group of patients. Navigation between different views, such as zooming in or out to different levels of detail granularity can be performed, e.g., via touchscreen or the like. Through the use of icons, the SPWL can provide optimizing information detail using limited screen space.

The present innovation finds application in healthcare administration, particularly with regard to patient worklist management therein. However, it will be appreciated that the described techniques may also find application in other worklist systems, other healthcare-providing scenarios, other organization techniques, and the like.

The number of smart phone applications (“apps”) available today is increasing exponentially, and more apps are available every day. The Smart phone has become a part of humans' daily lives more than the television and the internet. Smart phones are truly mobile and fit within one's pocket, as opposed to bulky laptops and tablets, which require two hands to operate.

By nature, a conventional multi-patient worklist application typically requires access to a large amount of data covering multiple patients. For instance, nurses in a general ward may care for 3 to 8 patients during the day shift, 6 to 15 patients during evenings, and up to 40 patients at night. Every patient has scheduled therapies and scheduled events. Therapies can include such tasks as medication administration and treatments. Scheduled events can include diagnostic tests, education sessions, and ADT activities. Some of these scheduled activities must be performed close to a scheduled time due to clinical requirements and are considered time sensitive. For example, pre-operative medications must be given close to one hour before the surgical procedure in order for the patient to be in the appropriate condition for surgery. The patients also may have ongoing therapies and conditions that require close monitoring such as intravenous lines using infusion pumps, surgical wounds, chest tubes, urinary catheters, oxygen therapy, and fall prevention precautions.

In caring for multiple patients, the general ward nurse is never in one place for long. Patients are found in multiple rooms. The nurse is often required to run from room to room, assessing and caring for patients. Ward nurses spend much less time with the patient than critical care nurses and more time traveling between patients. The ward nurses hands must be free to take blood pressures, give medications, assist patients in ambulation, and other tasks.

One challenge for nurses is how to care for multiple patients within a limited time period, carrying out the required tasks on time. When the nurse first comes onto a shift and reviews the assigned patients, the nurse begins to plan his or her day. The nurse must assess time-critical tasks as well as what health parameters to monitor on the patients in their limited time at the bedside. Much of this information is recorded in the nurse's paper worklist, which is created when the nurse first arrives at the healthcare facility for a shift. This static piece of paper is the lifeline for the general ward nurse and is frequently reviewed to plan patient care for the multiple patients assigned to the nurse. Even in hospitals with electronic health records and computerized worklists, the nurses often print out a copy of the worklist to carry in their pocket for quick and easy access to the patients' schedules and tasks. When a patient's care changes, such as when new physician orders are written, the nurse must physically go to the patient's chart and copy the orders that affect his or her shift onto the paper worklist. At the end of the shift, the nurse uses the paper worklist as the source of information for charting into the patient record. The worklist is the main document used by general ward nurses in caring for the patient.

The present application provides new and improved systems and methods that facilitate providing a smart-phone based multi-patient worklist that is mobile and easily operated with one hand, which overcome the above-referenced problems and others.

In accordance with one aspect, a computer-readable medium has stored thereon instructions for providing a smartphone-based multi-patient worklist (SPWL) for charting patient status information, the instructions comprising receiving user login information identifying a user of a smartphone presenting to the user on a user interface of the smartphone a worklist home screen comprising scheduled tasks to be completed during the user's shift for a plurality of patients assigned to the user, and detecting that the user has navigated to a patient details screen comprising tasks to be performed for a specific one of the plurality of patients. The instructions further comprise receiving user-input status update information regarding completion of the tasks to be performed for the specific patient, and transmitting the status update information to a central server for real-time updating of the medical chart of the specific patient.

According to another aspect, a personal communication device is configured to provide a smartphone-based multi-patient worklist (SPWL) for charting patient status information, and comprises a processor configured to receive user login information identifying a user of a smartphone, present to the user on a user interface of the smartphone a worklist home screen comprising scheduled tasks to be completed during the user's shift for a plurality of patients assigned to the user, and detect that the user has navigated to a patient details screen comprising tasks to be performed for a specific one of the plurality of patients. The processor is further configured to receive user-input status update information regarding completion of the tasks to be performed for the specific patient, and transmit the status update information to a central server for real-time updating of the medical chart of the specific patient.

According to another aspect, a method of providing a smartphone-based multi-patient worklist (SPWL) for charting patient status information comprises receiving user login information identifying a user of a smartphone, presenting to the user on a user interface of the smartphone a worklist home screen comprising scheduled tasks to be completed during the user's shift for a plurality of patients assigned to the user, and detecting that the user has navigated to a patient details screen comprising tasks to be performed for a specific one of the plurality of patients. The method further comprises receiving user-input status update information regarding completion of the tasks to be performed for the specific patient, and transmitting the status update information to a central server for real-time updating of the medical chart of the specific patient.

One advantage is that worklist mobility is improved.

Another advantage is that patient charting can be performed in real time without a need for a laptop or other bulky computing device.

Still further advantages of the subject innovation will be appreciated by those of ordinary skill in the art upon reading and understand the following detailed description.

The patent or application file contains at least one drawing executed in color. Copies of this patent or patent application publication with color drawing(s) will be provided by the Patent and Trademark Office upon request and payment of the necessary fee.

The drawings are only for purposes of illustrating various aspects and are not to be construed as limiting.

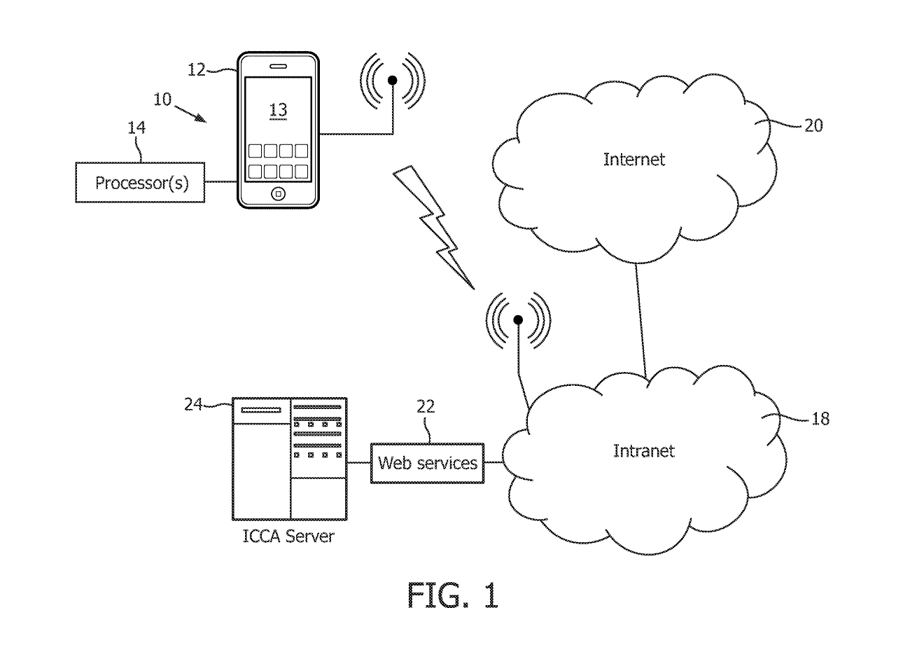

FIG. 1 illustrates a system that facilitates providing a smart phone based multi-patient worklist (SPWL) via a smartphone (or tablet).

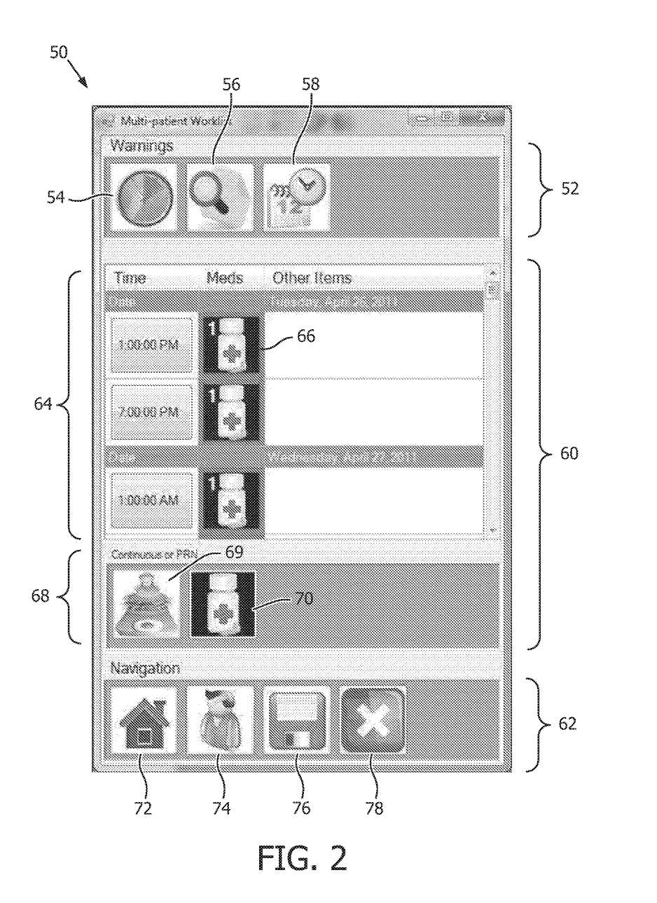

FIG. 2 shows an example of a “schedule” screen in the SPWL as presented on an Apple operating system, in accordance with one or more aspects described herein.

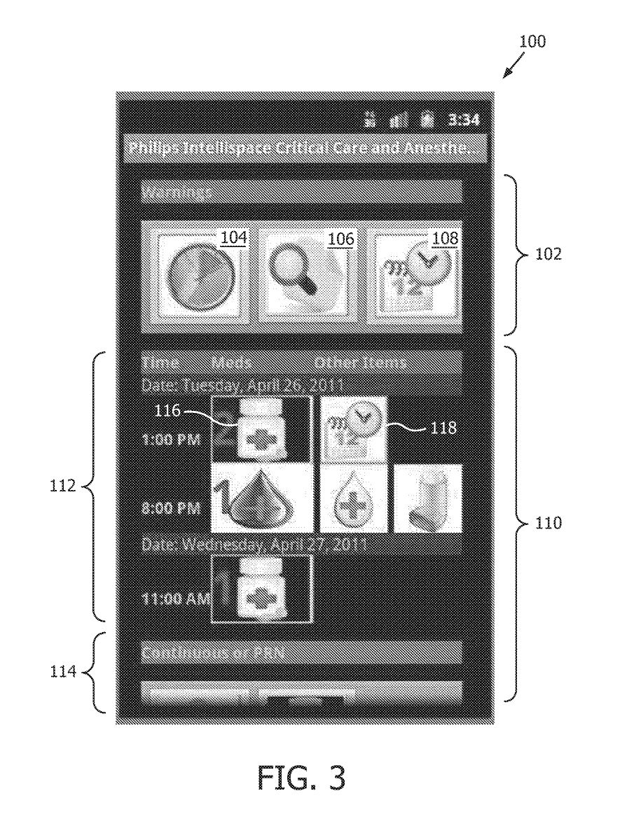

FIG. 3 shows an example of a “schedule” screen in the SPWL as presented on an Android operating system, in accordance with one or more aspects described herein.

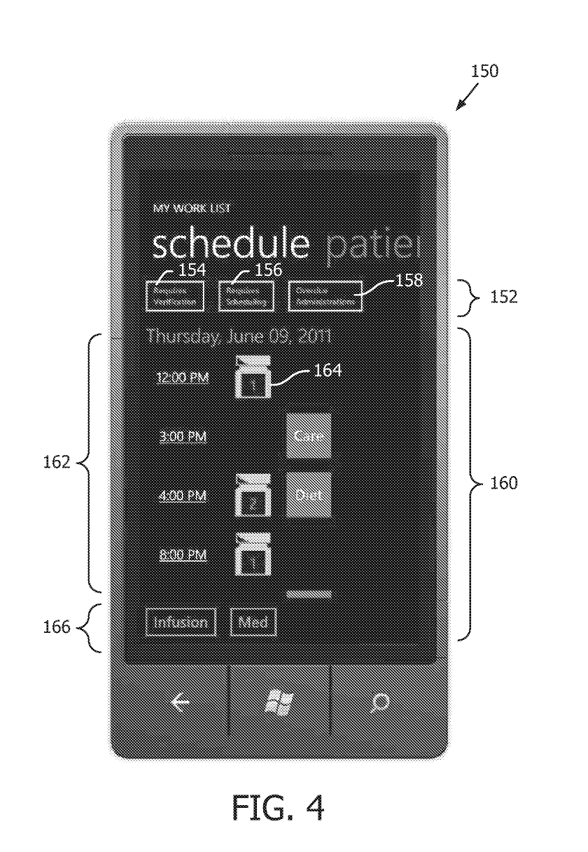

FIG. 4 shows an example of a “schedule” screen in the SPWL as presented on a Windows Phone operating system, in accordance with one or more aspects described herein.

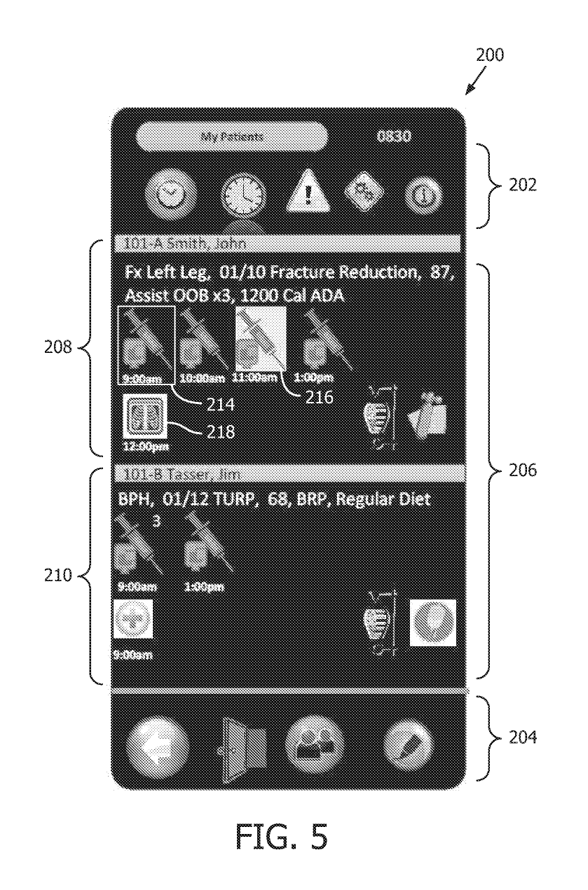

FIG. 5 shows an example of a “patients” screen in the SPWL as presented on a smart phone, in accordance with one or more aspects described herein.

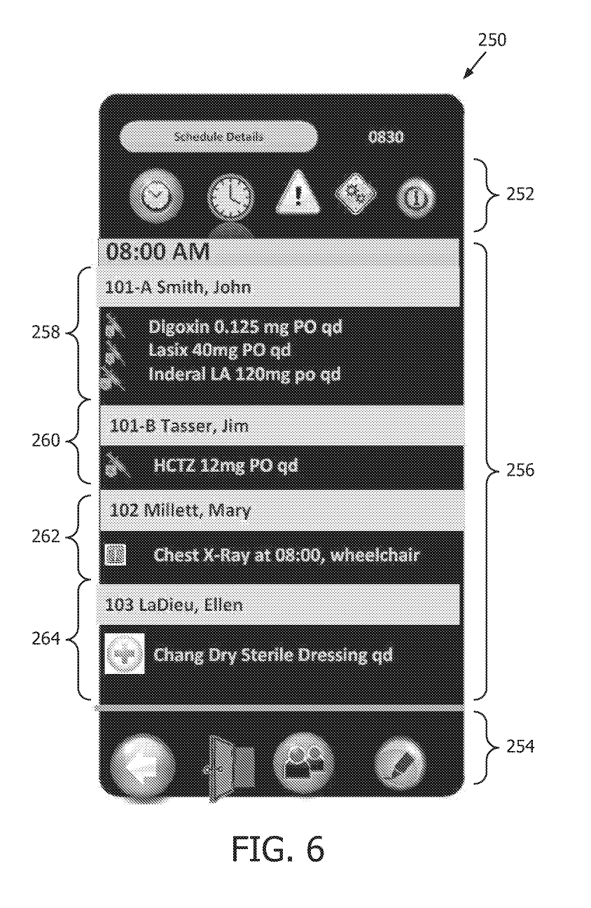

FIG. 6 shows an example of a “schedule details” screen in the SPWL as presented on a smart phone, in accordance with one or more aspects described herein.

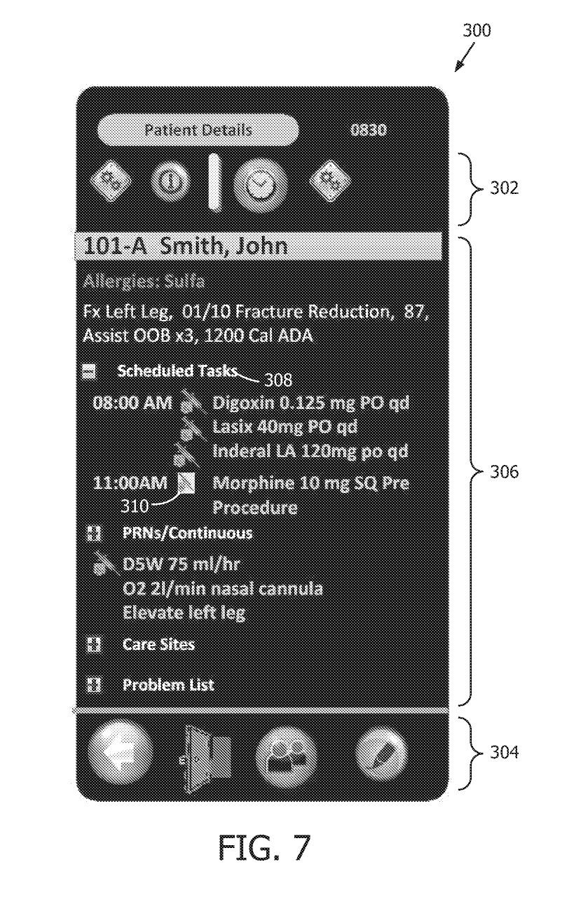

FIG. 7 shows an example of a “patient details” screen in the SPWL as presented on a smart phone, in accordance with one or more aspects described herein.

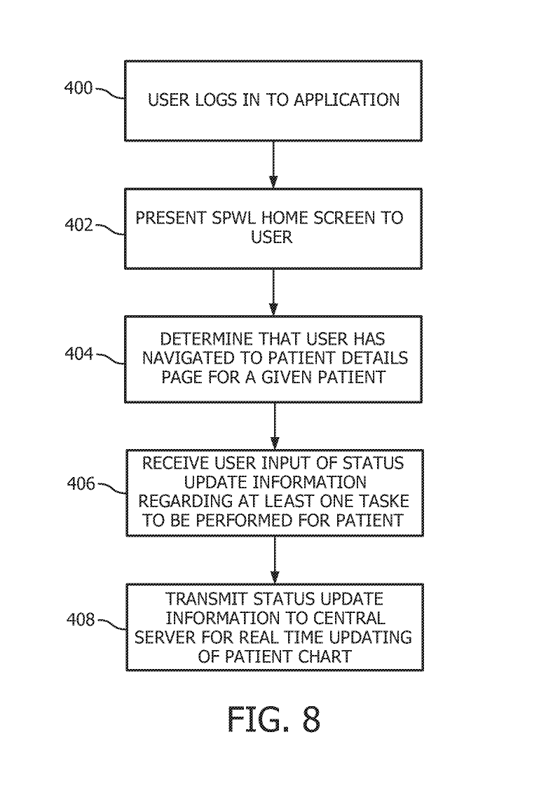

FIG. 8 illustrates a method for providing a smartphone-based multi-patient worklist (SPWL) for charting patient status information, in accordance with one or more aspects described herein.

The described systems and methods overcome the above-mentioned problems by providing a unique user interface that allows one to view their entire workload for the shift with the ability to drill down or zoom in to individual patients or time periods. Icons are used to visually condense extensive information across multiple patients. Examining the icons, a nurse or other clinician can quickly discern what key tasks require completion, as well as key patient conditions and therapies that require close monitoring and assessment. Icons are also used to inform the nurse of clinical advisories and workflow reminders. The worklist is automatically generated, dynamically updated, and can be used to chart on the patient record.

The described Smart Phone Based Multi-Patient worklist (SPWL) replaces the paper multi-patient worklist and incorporates clinical decision support and workflow reminders. The SPWL has a user interface approach that addresses the small screen size while still providing the nurse summarized and detailed views into the tasks that must be done and the patient concerns to be addressed. The SPWL has a set of screens that use icons to provide summary information about all the patients assigned to the nurse. Easy navigation from screen to screen allows one to view the information for multiple patients in various formats, focusing on different aspects of care. The SPWL is automatically generated based upon the patients assigned to the nurse, without requiring the nurse to generate a paper worklist. The SPWL automatically updates whenever changes are made in the patient's chart, without requiring the nurse to update the paper worklist. The worklist also provides reminders and warnings to the nurse concerning patient and workflow issues, which are not available on the paper worklist. As an application on the smart phone, the SPWL is easy to carry to any room to any situation, leaving the nurse's hands free for giving care.

Additionally, the nurse can use the SPWL to chart the tasks completed and on the patient assessments, which cannot be done on a conventional paper worklist. All charting performed via the SPWL automatically updates the patient's record. The SPWL obtains and sends information to clinical information systems, such as the Philips Intellivue Critical Care and Anesthesia system (ICCA). The ICCA can be used in the general ward at healthcare sites, and documentation is similar between critical care and general wards. One difference between the critical care units and the general ward is that on the general ward, nurses care for larger number of patients while the critical care nurses often care for 1 to 2 patients. Thus, the SPWL is an application that can support the use of the ICCA in the general ward clinical area.

FIG. 1 illustrates a system 10 that facilitates providing a smart phone based multi-patient worklist (SPWL) via a smartphone 12 (or tablet). The smartphone 12 includes a user interface 13, as well as a processor 14 that executes, and a memory 16 stores, computer-executable instructions for performing the various functions, methods, techniques, applications, etc., described herein. The smartphone communicates wirelessly (e.g., via a Wi-Fi connection, a cellular connection, a short range connection such as BlueTooth™, etc.) with an intranet 18, which is further coupled to an internet 20. Web services 22 are provided to the smartphone 12 from a server 24 (e.g., an ICCA server) via the intranet 18. Although depicted as communicating with the intranet 18, it will be appreciated that the smartphone can also access the intranet 18 via the internet 20.

It will be understood that the processor 14 executes, and the memory 16 stores, computer executable instructions for carrying out the various functions and/or methods described herein. The memory 16 may be a computer-readable medium on which a control program is stored, such as a disk, hard drive, or the like. Common forms of computer-readable media include, for example, floppy disks, flexible disks, hard disks, magnetic tape, or any other magnetic storage medium, CD-ROM, DVD, or any other optical medium, RAM, ROM, PROM, EPROM, FLASH-EPROM, variants thereof, other memory chip or cartridge, or any other tangible medium from which the processor 14 can read and execute. In this context, the system 10 may be implemented on or as one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphics processing unit (GPU), or PAL, or the like.

The web services 22 provide read and write access into an ICCA database (not shown) in the server 24. The web services 22 also provide object models using simple objects. The smartphone 12 employs native development language and tools to access the ICCA web services 22 and to develop a user interface for specific smartphone operating systems. The architecture thus facilitates providing a common level of functionality across varying smartphone operating systems based, e.g., on Android™ Windows Phone™, or Apple-based systems. By loosely coupling with the ICCA architecture through interface and objects, the user interface is scalable to any smart phones and any operating system.

For the Android mobile platform, the application is built using standard development tools such as, and without being limited to, the Eclipse integrated development environment (IDE) and the Java language, and uses the Android Java native API library. The application uses Google's model-view-controller (MVC) pattern and interacts with the ICCA server via Javascript Object Notation (.JSON) web services. In this embodiment, the Android device uses device persistence (e.g., SQL Lite) to provide optimum performance and cached data for off-line users.

For the Apple iPhone™ mobile platform, the application is built using Apple iOS development tools such as, by way of example and without being limited to, the Xcode integrated development environment (IDE), Interface Builder, and the iPhone software development kit (SDK). The user interface 13 is defined using the iPhone framework, e.g., Cocoa Touch™. The use of an iPhone framework such as Cocoa Touch ensures that the application has the look and feel of other iPhone applications. Objective-C programming can be used to define the business logic, manage the screen to screen navigation and to access the ICCA Web Service to retrieve the patient worklist information for display. The data returned from the International Conference on Image Processing (ICIP) Web Service via an extensible markup language (.XML) or Javascript Object Notation (.JSON) structure is parsed using either the native NSXML Parser or a third party JSON parser.

For the Microsoft Windows Phone mobile platform, the application is built using the Windows Phone Software Development Kit. The user interface 13 is created using the extensible application markup language (XAML) markup language. The application main screen is implemented using the Windows Phone Pivot Control. This allows a user to navigate between the “My Schedule” and “My Patients” screens (FIGS. 2-5) using, e.g., the Windows Phone screen gesture. A “Schedule Details” (FIG. 5) screen is displayed when a user selects any time-based row on the “My Schedule” screen (FIGS. 2-4). A “Patient Details” screen (FIG. 7) is displayed when a user selects a patient row on the “My Patients” screen (FIG. 6). Business logic and networking features are implemented using, e.g., the C# programming language, or any other suitable programming language. The application communicates with the ICCA web services 22 using the WebClient and HttpWebRequest classes provided by the Windows Phone runtime environment.

The application presented via the user interface 13 is distributed via the phone manufacturer's application store and/or can be manually installed by executing an installation package on the smartphone 12. The package can downloaded to the device via a direct connection between a PC (not shown) and the smartphone 12, downloaded from a web screen, or sent to users via e-mail.

FIGS. 2-7 show a screenshots of examples of SPWL screens or screens as presented via the user interface 13 (FIG. 1) to a nurse or other user, in accordance with one or more aspects presented herein. The user interface 13 presents multi-patient summary screens using icons to communicate a large amount of information using the limited screen space of the smartphone. Icons represent categories or type of tasks, warnings, therapies, concerns, etc. A single icon can represent one or a plurality of items. If there is more than one item represented by an icon, the icon has a number displayed with it. The icons of the user interface screens or screens described herein permit a user to “drill down” or “zoom” into more detail about any patient, item, or scheduled time range, as well as to zoom out to a bigger picture. Icons presented to the user are dynamically updated when the patient record changes, and present ICCA clinical advisories and work flow reminders for clinical decision support. The icons are automatically generated and/or updated once the user identifies one or more patients assigned to the nurse.

Additionally, the described SPWL screens or screens permit the user to chart completed tasks and simple assessment information. All charting information is automatically communicated to a patient's chart, and the patient's chart is updated in real time. All communication between the smart phone and clinical information system follows the security and privacy requirements for HIPAA and Meaningful Use.

The various screens presented to a user via the SPWL user interface can include without limitation: a “schedule” screen 50, 100, 150 (FIGS. 2-4), which is a time based screen showing the tasks to done by time for multiple patients; a “patients” screen 200 (FIG. 5), which is a patient-based screen that shows details about the group of patients assigned to the nurse or other user; a “schedule detail” screen 250 that focuses on what needs to be done for a particular time period. A read-only screen; and a “patient details” screen 300 that focuses on the details of a particular patient. This screen allows one to chart information on the patient. In one embodiment, only patient details screen 300 can be charted on as a safety feature in order to mitigate a situation where the nurse inadvertently charts that a task has been completed or a medication has been administered in an incorrect patient's chart.

FIG. 2 shows an example of a “my schedule” screen 50 in the SPWL as presented on an Apple operating system, in accordance with one or more aspects described herein. The schedule screen 50 is a time based screen showing the tasks to done by time for multiple patients. In one embodiment, the schedule screen 50 is a read-only screen to prevent inadvertent modification of information presented therein. The schedule screen 50 is a time oriented view of the tasks to be done for a time period across the patients assigned to the nurse. The schedule screen is an initial screen that is displayed upon login. In one embodiment, the schedule screen is presented as a time based grid with the most urgent task or time presented first, and tasks that are further off in time presented further down the screen. In another embodiment, only time periods that have schedule tasks are displayed.

The schedule screen 50 includes a warnings panel 52 at the top of the screen that shows icons for notifications 54 (e.g., new lab results are available) that require verification, clinical advisories 56 (e.g., notifications that a patient's condition has changed, such as a drop in blood pressure, etc.) for one or multiple patients, and workflow reminders 58 (e.g., time-critical task reminders that are impending or overdue). A main screen area 60 includes variable content depending on the screen purpose/content. A navigation panel 62 at the bottom of the screen allows the user to navigate to other screens, return to a previous screen, log-out, and sign-in, etc.

The main screen area 60 includes a scheduled time row area 64 showing scheduled items by time. In one embodiment, medications are presented as higher priority than other items. A medication icon 66 includes information related the number of medication administrations due for the scheduled time or time period across all patients assigned to the nurse. In the illustrated example, the user is presented with medication icons that indicate that medications must be given to one or more patients at 1 pm and 7 pm on April 26, and at 1 am on April 27.

Different icons are employed to represent, e.g., medication administrations, scheduled surgeries and procedures, tests (e.g., radiology, blood panels, etc.), ADT actions (e.g., admissions, discharges, transfers, etc.), treatments (e.g., dressing changes, etc.), and miscellaneous items. In one embodiment, icons with time sensitive tasks are highlighted, e.g., in yellow or some other predetermined color. In another embodiment, rows with items that are overdue are presented in, e.g., red text or some other suitable manner. Once a task is charted as done/held, it is removed from the schedule.

A “non-scheduled task” row 68 shows icons 69, 70 for types of tasks that are not scheduled and have no associated times. Non-scheduled tasks comprise icons representing key concerns or conditions to note. A different icon represents each category of tasks (e.g., continuous or ongoing tasks, pro re nata (PRN) or “as needed” tasks, etc.). Examples of such tasks include, e.g., infusions including drips, total parenteral nutrition (TPN), colloids, blood transfusions, intravenous infusions, drains and output sites, wounds/surgical sites, ongoing respiratory treatments such as oxygen therapy/ventilators, and other miscellaneous information. In the illustrated example, a “diet” icon 69 represents a dietary task the user is to perform, such as checking that the patient consumed specific foods or a certain number of calories. A medication icon 70 is also shown, which represents an ongoing medication task that the user is to perform.

The navigation panel 62 includes a “home” icon 72 that a user selects to be returned to the SPWL home screen, a “patients” icon 74 that the user selects to view the “my patients” screen, a “save” icon 76 that a user selects to save data, and a “return” icon 78 that a user selects to return to a previous screen. Actions that a user can perform on the schedule screen 50 include, without limitation, selecting any time-based row in order to navigate to the schedule details screen (FIG. 6) for that time period, as well as selecting any task icon in order to navigate to the schedule details filtered by that time period and/or filtered by that type of task.

FIG. 3 shows an example of a “my schedule” screen 100 in the SPWL as presented on an Android operating system, in accordance with one or more aspects described herein. The schedule screen 100 is a time based screen showing the tasks to done by time for multiple patients. In one embodiment, the schedule screen 100 is a read-only screen to prevent inadvertent modification of information presented therein. The schedule screen 100 is a time oriented view of the tasks to be done for a time period across the patients assigned to the nurse. The schedule screen is an initial screen that is displayed upon login. In one embodiment, the schedule screen is presented as a time based grid with the most urgent task or time presented first, and tasks that are further off in time presented further down the screen. In another embodiment, only time periods that have schedule tasks are displayed.

The schedule screen 100 includes a warnings panel 102 at the top of the screen that shows icons for notifications 104 (e.g., new lab results are available) that require verification, clinical advisories 106 (e.g., e.g., test or procedures that require scheduling, etc.) for one or multiple patients, and workflow reminders 108 (e.g., time-critical task reminders that are impending or overdue). A main screen area 110 includes variable content depending on the screen purpose/content. A navigation panel not shown at the bottom of the screen allows the user to navigate to other screens, return to a previous screen, log-out, and sign-in, etc.

The main screen area 110 includes a scheduled time row 112 showing scheduled items. In one embodiment, medications are presented as higher priority than other items. A medication icon 112 includes information related the number of medication administrations due for the scheduled time or time period across all patients assigned to the nurse. In the illustrated example, the user is presented with icons that indicate that two medications must be given, and one time-critical task must be performed, for one or more patients at 1 pm on April 26. At 8 pm on April 26, additional tasks must be performed, and at 11 am on April 27, medication is to be administered to a patient.

Different icons are employed to represent, e.g., medication administrations, scheduled surgeries and procedures, tests (e.g., radiology, blood panels, etc.), ADT actions (e.g., admissions, discharges, transfers, etc.), treatments (e.g., dressing changes, etc.), and miscellaneous items. In one embodiment, icons with time sensitive tasks are highlighted, e.g., in yellow or some other predetermined color. In another embodiment, rows with items that are overdue are presented in, e.g., red text or some other suitable manner. Once a task is charted as done/held, it is removed from the schedule.

A “non-scheduled task” row 114 can include icons (not shown in FIG. 3) for types of tasks that are not scheduled and have no associated times. Non-scheduled tasks comprise icons representing key concerns or conditions to note. A different icon represents each category of tasks (e.g., continuous or ongoing tasks, pro re nata (PRN) or “as needed” tasks, etc.). Examples of such tasks include, e.g., infusions including drips, total parenteral nutrition (TPN), colloids, blood transfusions, intravenous infusions, drains and output sites, wounds/surgical sites, ongoing respiratory treatments such as oxygen therapy/ventilators, and other miscellaneous information. For example, a “diet” icon represents a dietary item the user is to perform, such as checking that the patient consumed specific foods or a certain number of calories. A medication icon represents an ongoing medication task that the user is to perform. It will be understood that the icons of the non-scheduled task row 114 of FIG. 3 present information to the user in a manner similar to that described with regard to FIG. 2.

A user can scroll down to a navigation panel (not shown in FIG. 3) includes icons similar to those described with regard to FIG. 2, such as a “home” icon that a user selects to be returned to the SPWL home screen, a “patients” icon that the user selects to view the “my patients” screen, a “save” icon that a user selects to save data, and a “return” icon that a user selects to return to a previous screen. Actions that a user can perform on the schedule screen 100 include, without limitation, selecting any time-based row in order to navigate to the schedule details screen (FIG. 6) for that time period, as well as selecting any task icon in order to navigate to the schedule details filtered by that time period and/or filtered by that type of task.

FIG. 4 shows an example of a “schedule” screen 150 in the SPWL as presented on a Windows Phone operating system, in accordance with one or more aspects described herein. The schedule screen 150 is a time based screen showing the tasks to done by time for multiple patients. In one embodiment, the schedule screen 150 is a read-only screen to prevent inadvertent modification of information presented therein. The schedule screen 150 is a time oriented view of the tasks to be done for a time period across the patients assigned to the nurse. The schedule screen is an initial screen that is displayed upon login. In one embodiment, the schedule screen is presented as a time based grid with the most urgent task or time presented first, and tasks that are further off in time presented further down the screen. In another embodiment, only time periods that have schedule tasks are displayed.

The schedule screen 150 includes a warnings panel 152 at the top of the screen that shows icons for notifications 154 (e.g., new lab results are available) that require verification, clinical advisories 156 (e.g., e.g., test or procedures that require scheduling, etc.) for one or multiple patients, and workflow reminders 158 (e.g., time-critical task reminders that are impending or overdue). A main screen area 160 includes variable content depending on the screen purpose/content. A navigation panel (not shown) at the bottom of the screen allows the user to navigate to other screens, return to a previous screen, log-out, and sign-in, etc.

The main screen area 160 includes a scheduled time row 162 showing scheduled items. In one embodiment, medications are presented as higher priority than other items. A medication icon 164 includes information related the number of medication administrations due for the scheduled time or time period across all patients assigned to the nurse. In the illustrated example, the user is presented with icons that indicate that one medication must be given at 12 pm, two medications given at 4 pm, and another medication must be given at 8 pm. Additionally, at 3 pm a patient care task must be completed, and at 4 pm a diet-related task requires completion.

Different icons are employed to represent, e.g., medication administrations, scheduled surgeries and procedures, tests (e.g., radiology, blood panels, etc.), ADT actions (e.g., transfers, etc.), treatments (e.g., dressing changes, etc.), and miscellaneous items. In one embodiment, icons with time sensitive tasks are highlighted, e.g., in yellow or some other predetermined color. In another embodiment, rows with items that are overdue are presented in, e.g., red text or some other suitable manner. Once a task is charted as done/held, it is removed from the schedule.

A “non-scheduled task” row 166 can include icons (not shown in FIG. 3) for types of tasks that are not scheduled and have no associated times. Non-scheduled tasks comprise icons representing key concerns or conditions to note. A different icon represents each category of tasks (e.g., continuous or ongoing tasks, PRN tasks, etc.). Examples of such tasks include, e.g., infusions including drips, TPN, colloids, blood transfusions, intravenous infusions, drains and output sites, wounds/surgical sites, ongoing respiratory treatments such as oxygen therapy/ventilators, and other miscellaneous information. For example, a “diet” icon represents a dietary item the user is to perform, such as checking that the patient consumed specific foods or a certain number of calories. A medication icon represents an ongoing medication task that the user is to perform. It will be understood that the icons of the non-scheduled task row 166 of FIG. 3 present information to the user in a manner similar to that described with regard to FIG. 2.

A user can scroll down to a navigation panel (not shown in FIG. 3) includes icons similar to those described with regard to FIG. 2, such as a “home” icon that a user selects to be returned to the SPWL home screen, a “patients” icon that the user selects to view the “my patients” screen, a “save” icon that a user selects to save data, and a “return” icon that a user selects to return to a previous screen. Actions that a user can perform on the schedule screen 150 include, without limitation, selecting any time-based row in order to navigate to the schedule details screen (FIG. 6) for that time period, as well as selecting any task icon in order to navigate to the schedule details filtered by that time period and/or filtered by that type of task.

FIG. 5 shows an example of a “my patients” screen 200 in the SPWL as presented on a smart phone, in accordance with one or more aspects described herein. The patients screen 200 comprises a warning panel 202 and a navigation panel 204, which comprise icons similar to those described with regard to the preceding figures. A main screen area 206 shows selectable entries 208, 210 for each patient assigned to the nurse in order of the bed census. The nurse selects a patient to view the worklist information associated with the selected patient in greater detail.

In the illustrated example, patients John Smith and Jim Tasser are depicted. Each patient entry 208, 210 comprises demographic information such as patient name, room number, main diagnoses, surgery and surgery date. Each patient entry further comprises a list of medications due for next 4 hours, using icons for each medication. Each medication icon 212 displays the time the medication is to be administered. In the depicted example, a time sensitive dose 214 is highlighted. In one embodiment, overdue medications are depicted in a predetermined color (e.g., red or the like). Each patient entry also comprises a list of other scheduled tasks using icons that represent that type of task with their scheduled time. For instance, and x-ray icon 216 indicates that John Smith is scheduled for a procedure in radiology at 12 pm. Non-scheduled concerns are also represented by icons, where icon can represent one or a plurality of concerns. For example, an IV icon 218 indicates to the user that the patients' IV bags should be checked.

FIG. 6 shows an example of a “schedule details” screen 250 in the SPWL as presented on a smart phone, in accordance with one or more aspects described herein. The schedule details screen 250 comprises a warning panel 252 and a navigation panel 254, which comprise icons similar to those described with regard to the preceding figures. A main screen area 256 shows selectable patient rows 258, 260, 262, 264 for each patient assigned to the nurse in order of a bed census.

The schedule details screen 250 provides details for the patients and their required tasks for a particular scheduled time and/or type of task. Complete information about the task is provided such as drug dose or type of infusion. When a particular category of task is selected, then all other types of tasks are filtered out of the displayed interface (i.e., the user drills down to the selected task). When a particular time period is selected, then all other time periods are filtered out of the displayed interface (i.e., the user drills down to the selected time period). In one embodiment, rows of information are organized by patient room, such as according to how a bed census for the healthcare facility is listed. Each patient row lists one or a plurality of tasks due for that time (if time based) of that task category (when a particular icon is selected.) In one embodiment, the schedule details screen is a read-only screen to mitigate inadvertent changes to the displayed data. Each patient row contains demographic information such as name, room, diagnoses, age, allergies, surgery and surgical date, etc. Upon selecting a patient, the user is presented with a patient details screen (FIG. 7).

FIG. 7 shows an example of a “patient details” screen 300 in the SPWL as presented on a smart phone, in accordance with one or more aspects described herein. The patient details screen is a “deepest” screen of the plurality of SPWL screens in that the user drills down to the patient details screen through one or more other screens (e.g., the “my patients” screen or the like), such that the user cannot navigate beyond the patient details screen but is permitted to navigate back through the plurality of screens. Additionally, in contrast to the other SPWL screens, which are read-only to prevent inadvertent charting thereon, the patient details screen can be written to so that the user can perform charting thereon.

The patient details screen 300 comprises a warning panel 302 and a navigation panel 304, which comprise icons similar to those described with regard to the preceding figures. A main screen area 306 shows the complete task information related to tasks to be performed for the patient organized by time. In the depicted example, a “scheduled tasks” icon 308 has been expanded in the main screen area 306 on the Patient Details screen for John Smith, as indicated by the “-” next to the icon 308. The scheduled tasks for John Smith include four medications: three medications to be administered at 8 am and a fourth to be administered at 11 am, which is time critical as indicated by the highlighted medication icon 310. ON this screen (the Patient Details screen), the user is permitted to select a given task, and upon completion thereof, input an indication that the task has been completed. The user input is used to update the patient's chart in real time.

The Patient Details screen also shows three “+” sign icons next to respective icons/headings that can be expanded upon selection of the corresponding “+” sign icon. The respective headings for the “+” signs correspond to continuous infusions/therapies and PRNs, care sites, problem list, and can further comprise patient detailed information. A user clicks on the “+” icon to expand the respective content. The user then selects a task under the expanded heading and inputs an indication regarding whether the task has been completed. In one embodiment, a short comment may be entered, e.g., via the smartphone keyboard or via a microphone on the smartphone device. When charting is done, or “held,” other information may be entered such as medication dose.

With regard to the screens of the SPWL as discussed with regard to FIGS. 2-7, in one embodiment, the warnings panel of the herein-described SPWL screens is displayed at the top of every screen. The warning panel comprises one or more warning icons representing different types patient and workflow warnings. Types of warnings can include overdue workflow reminders, which indicate that a scheduled tasks is more than a predetermined amount of time (e.g., 30 minutes, etc.) overdue. The overdue workflow reminder icons can be highlighted in a first predetermined color (e.g., red, etc.). The warning panel can also include time sensitive interventions that are due within a predetermined time period (e.g., the next 30 minutes, etc.). Time sensitive interventions are scheduled tasks that must be done close to the schedule time, and these icons can be highlighted in a second predetermined color (e.g., yellow, etc.). Clinical advisories, such as a warning that the patient's potassium (K) value is too low can also be included in the warning panel.

Additionally, the warning panel can include icons for workflow reminders, such as the arrival of new items for the patient or the like, and notifications that new laboratory results are available for an assigned patient. Selecting an icon in the warning panel displays warning details including patient name and room. When the user is on a patient-specific screen, the warnings panel can show warnings both for the specific patient and for all assigned patients, separated by a visual indicator. This allows the nurse to be apprised of all patients' status even the nurse is focusing on a specific patient.

The Navigation Panel is displayed at the bottom of the SPWL screens, according to one embodiment. The Navigation Panel comprises selectable icons to access the following the My Patients screen, the My Schedule screen, or to return to a previous screen, as well as to log out or log in (e.g., by entering password and sending login information to the clinical information system). This feature allows the nurse to chart multiple items with a single entry of password. It will be appreciated that in another embodiment, the warnings panel is displayed at the bottom of the SPWL screens and the navigation panel is displayed at the top of the SPWL screens.

FIG. 8 illustrates a method for providing a smartphone-based multi-patient worklist (SPWL) for charting patient status information, in accordance with one or more aspects described herein. At 400, user login information identifying a user of a smartphone or other personal communication device is received. For, instance, the user may enter his or her login credentials, and the smartphone communicates with a central server to verify the user's credentials. In another embodiment, the user's credentials are verified locally by the smartphone. Once the user has logged in, a worklist home screen is presented to the user on a user interface of the smartphone, at 402. The worklist home screen comprises scheduled tasks to be completed during the user's shift for a plurality of patients assigned to the user. In one embodiment, the home screen is a “My Schedule” screen such as is described with regard to FIGS. 2-4. In another embodiment, the home screen comprises selectable icons via which the user can navigate to any of the screens described with regard to FIGS. 2-7. The home screen may also include the herein-described warnings panel and navigation panel.

At 404, a determination is made that the user has navigated to a patient details screen comprising tasks to be performed for a specific one of the plurality of patients. At 406, user-input status update information is received regarding completion of the tasks to be performed for the specific patient. For instance, the user may indicate that a time critical medication was given at an appointed time, or that a patient's IV bag was changed. The status update information is transmitted, at 408, to a central server for real-time updating of the medical chart of the specific patient. In this manner, charting is performed by the user (e.g., a nurse) on the Patient Details screen, while other screens of the SPWL application are provided as read-only to prevent inadvertent status updating.

The innovation has been described with reference to several embodiments. Modifications and alterations may occur to others upon reading and understanding the preceding detailed description. It is intended that the innovation be construed as including all such modifications and alterations insofar as they come within the scope of the appended claims or the equivalents thereof.

1. A computer-readable medium having stored thereon instructions for providing a smartphone-based multi-patient worklist (SPWL) for charting patient status information, the instructions comprising:
receiving user login information identifying a user of a smartphone;
presenting to the user on a user interface of the smartphone a worklist home screen comprising icons that represent scheduled tasks to be completed during the user's shift for a plurality of patients assigned to the user;
detecting that the user has navigated to a patient details screen comprising tasks to be performed for a specific one of the plurality of patients;
receiving user-input status update information regarding completion of the tasks to be performed for the specific patient; and
transmitting the status update information to a central server for real-time updating of the medical chart of the specific patient wherein at least one of:
the patient details screen is a deepest screen of a plurality of screens presented to the user via the SPWL, such that the user cannot navigate beyond the patient details screen but is permitted to navigate back through the plurality of screens; and
the plurality of screens comprises a patients screen, which is a read-only screen that comprises icons which graphically present to the user a summary of details related to multiple patients assigned to the user.

2. (canceled)

3. The computer-readable medium according to claim 1, wherein the user is permitted to chart patient information for a given patient only on the patient details screen corresponding to the given patient.

4. The computer-readable medium according to claim 1, wherein the plurality of screens comprises a schedule screen which is a time-based, read-only screen that comprises icons which graphically present to the user a summary of scheduled tasks for the user to perform for multiple patients.

5. The computer-readable medium according to claim 1, wherein the plurality of screens comprises a patients screen, which is a read-only screen that comprises icons which graphically present to the user a summary of details related to multiple patients assigned to the user.

6. The computer-readable medium according to claim 1, wherein the plurality of screens comprises a schedule details screen, which is a time-based, read-only screen that comprises icons which graphically present to the user a summary of scheduled tasks for the user to perform during one or more selected time periods.

7. The computer-readable medium according to claim 1, wherein the summarized information comprises at least one of tasks to be performed for the patient, medications to be administered to the patient, time-critical tasks or medications, and clinical procedures to be performed on the patient.

8. The computer-readable medium according to claim 1, wherein the home screen and the plurality of screens each include a warning panel that comprises selectable icons associated with one or more of overdue workflow reminders, time-critical tasks, clinical advisories, and laboratory result notifications, for all patients assigned to the user.

9. The computer-readable medium according to claim 1, wherein the home screen and the plurality of screens each include a navigation panel that comprises a plurality of selectable navigation icons that, when selected, respectively present the user with the patients screen, the schedule screen, a previous screen, a login screen, and a log out screen.

10. The computer-readable medium according to claim 1, wherein the icons further represent information related to at least one of:
one or more non-scheduled ongoing therapies;
one or more physical sites that require attention on one or more patients; and
one or more time-sensitive tasks that require performance within a predefined time window.

11. (canceled)

12. A personal communication device configured to provide a smartphone-based multi-patient worklist (SPWL) for charting patient status information, comprising:
a processor configured to:
receive user login information identifying a user of a smartphone;
present to the user on a user interface of the smartphone a worklist home screen comprising scheduled tasks to be completed during the user's shift for a plurality of patients assigned to the user;
detect that the user has navigated to a patient details screen comprising tasks to be performed for a specific one of the plurality of patients;
receive user-input status update information regarding completion of the tasks to be performed for the specific patient; and
transmit the status update information to a central server for real-time updating of the medical chart of the specific patient
wherein at least one of:
the patient details screen is a deepest screen of a plurality of screens presented to the user via the SPWL, such that the user cannot navigate beyond the patient details screen but is permitted to navigate back through the plurality of screens; and
the plurality screens comprises a patients screen, which is a read-only screen that comprises icons which graphically present to the user a summary of details related to multiple patients assigned to the user,

13. (canceled)

14. The personal communication device according to claim 12, wherein the user is permitted to chart patient information for a given patient only on the patient details screen corresponding to the given patient.

15. The personal communication device according to claim 12, wherein the plurality of screens comprises a schedule screen which is a time-based, read-only screen that comprises icons which graphically present to the user a summary of scheduled tasks for the user to perform for multiple patients.

16. (canceled)

17. The personal communication device according to claim 12, wherein the plurality of screens comprises a schedule details screen, which is a time-based, read-only screen that comprises icons which graphically present to the user a summary of scheduled tasks for the user to perform during one or more selected time periods.

18. The personal communication device according to claim 12, wherein the summarized information comprises at least one of tasks to be performed for the patient, medications to be administered to the patient, time-critical tasks or medications, and clinical procedures to be performed on the patient.

19. The personal communication device according to claim 12, wherein the home screen and the plurality of screens each include a warning panel that comprises selectable icons associated with one or more of overdue workflow reminders, time-critical tasks, clinical advisories, and laboratory result notifications, for all patients assigned to the user.

20. The personal communication device according to claim 12, wherein the home screen and the plurality of screens each include a navigation panel that comprises a plurality of selectable navigation icons that, when selected, respectively present the user with the patients screen, the schedule screen, a previous screen, a login screen, and a log out screen; and
the icons further represent information related to at least one of:
one or more non-scheduled ongoing therapies;
one or more physical sites that require attention on one or more patients; and
one or more time-sensitive tasks that require performance within a predefined time window.

21. (canceled)

22. (canceled)

23. (canceled)
